(19) United States
(12) Patent Application Publication
(10) Pub. No.: US 2025/0360150 A1
(43) Pub. Date: Nov. 27, 2025

(54) METHODS OF USING NEUROPLASTIC AGENTS AND TERRAFORMING MARS

(71) Applicant: Molly Caroline Brannock Metcalf, Boulder, CO (US)

(72) Inventor: Molly Caroline Brannock Metcalf, Boulder, CO (US)

(21) Appl. No.: 19/217,691

(22) Filed: May 23, 2025

Related U.S. Application Data
(60) Provisional application No. 63/651,806, filed on May 24, 2024.

(51) Int. Cl. A61K 31/675; A01G 15/00; A61K 31/137; A61K 31/36; A61K 31/4045; A61K 31/48; A61N 2/00

(52) U.S. Cl. CPC A61K 31/675; A01G 15/00; A61K 31/137; A61K 31/36; A61K 31/4045; A61K 31/48; A61N 2/006

(57) ABSTRACT

Various aspects of this disclosure relate to methods to treat health conditions in human patients, comprising administering a therapeutically-effective dose of a neuroplastic agent. Various aspects of this disclosure relate to method to assimilate human patients to prosthetics, comprising administering a therapeutically-effective dose of a neuroplastic agent. In some embodiments, the neuroplastic agent is a tryptamine such as psilocybin or psilocin.

### CROSS-REFERENCE TO RELATED APPLICATIONS

This patent claims priority to U.S. Provisional Patent Application No. 63/651,806, filed May 24, 2024, which is incorporated by reference in its entirety.

### BACKGROUND

Prosthetic devices are becoming increasingly sophisticated, and many modern devices function intuitively. A learning curve nevertheless exists for assimilation to new prosthetics. Methods to increase the rate of assimilation are desirable.

The CDC reports that almost 20 percent of married women of child-bearing age struggle to conceive. Infertility afflicts women at increasingly higher rates with age and is becoming a significant issue, particularly for educated women. The average age of mothers at first birth is now about 30 years for women with at least a college degree. First-time mothers are also older in many cities. In San Francisco County, for example, the average college-educated woman first becomes pregnant at 33 years. Improved methods to treat infertility are desirable.

Nonalcoholic fatty liver disease (NAFLD) is one of the fasting growing diseases in the world. About 25 percent of people have NAFLD worldwide, and about 5 percent have a more advanced form of this disease nonalcoholic steatohepatitis or NASH. As lipids accumulate around the liver of patients with NAFLD, about 20 percent will develop NASH within three to seven years, and 9-25 percent of these individuals will develop cirrhosis over the next ten to twenty years. In addition to the accumulation of lipids around the liver, NASH results in inflammation of the liver tissue and leads to scarring and fibrosis. In advanced stages, NASH can eventually develop into cirrhosis, liver failure, and/or cancer.

Current treatments for less advanced cases of NAFLD include weight loss, avoiding alcohol, and managing comorbidities such as diabetes. NAFLD often displays unremarkable symptoms in its early stages including fatigue, malaise, or pain in the general upper right quadrant of the abdomen. In more advanced stages, symptoms worsen and can include jaundice, ascites, esophageal varices, insulin resistance, muscle loss, itchy skin, red palms, enlarged spleen, swollen legs, and shortness of breath. Treatment options at these stages become increasingly limited, and, in the case of liver failure, liver transplants may be necessary. The United States liver transplant waitlist contains approximately 17,000 individuals, and patients who present with other chronic health conditions are typically ineligible. Comorbidities such as cardiovascular disease, diabetes, and metabolic syndrome, which typically also present with hypertension, hypercholesterolemia, and high triglycerides, often complicate the condition.

Insulin resistance is one of the serious complications of liver disease, and advanced stages can result in an overproduction of glucose and exacerbate progression of the disease. Pharmaceuticals based on glucagen-like-peptide-1 (GLP-1) show promise at helping patients lose weight, and increasing evidence suggests that these GLP-1 receptor agonists can potentially improve NAFLD by reducing inflammation, degree of steatosis, and even fibrosis. After several cases of “Ozempic psychosis” and self-hurting were reported, newly-appreciated side effects began to be recognized including insomnia, anxiety, depression, and other mental health related issues.

Improved methods to treat NAFLD, NASH, and other liver conditions are desirable.

### SUMMARY

Various aspects of this disclosure relate to a method to treat a condition in a human patient, comprising administering a therapeutically-effective dose of the neuroplastic agent to the human patient, wherein the therapeutically-effective dose of the neuroplastic agent is therapeutically effective to treat the condition or a symptom thereof.

The term “to treat” refers to at least one of: to cure a health condition; to increase the probability that a health condition will be cured; to shorten the time over which a health condition is cured; to increase the probability that the time necessary to cure a health condition will be shortened; to decrease the severity of a health condition; to increase the probability that the severity of a health condition will decrease; to shorten the time over which the severity of a health condition is decreased; to increase the probability that the time necessary to decrease the severity of a health condition will be shortened; to inhibit a health condition from worsening; to increase the probability that a health condition will not worsen; to delay the worsening of a health condition; to increase the probability that the worsening of a health condition will be delayed; to inhibit the occurrence or recurrence of a health condition; to decrease the probability that a health condition will occur or reoccur; to delay the onset of a health condition; to increase the probability that the onset of a health condition will be delayed; to alleviate at least one symptom of a health condition; to increase the probability that at least one symptom of a health condition will be alleviated; to shorten the time over which at least one symptom of a health condition is alleviated; to increase the probability that the time necessary to alleviate at least one symptom of a health condition will be shortened; to decrease the severity of at least one symptom of a health condition; to increase the probability that the severity of at least one symptom of a health condition will be decreased; to shorten the time over which the severity of at least one symptom of a health condition is decreased; to increase the probability that the time necessary to decrease the severity of at least one symptom of a health condition will be shortened; to inhibit at least one symptom of a health condition from worsening; to increase the probability that at least one symptom of a health condition will not worsen; to delay the worsening of at least one symptom of a health condition; to increase the probability that the worsening of at least one symptom of a health condition will be delayed; to inhibit at least one symptom of a health condition from occurring or reoccurring; to decrease the probability that at least one symptom of a health condition will occur or reoccur; to delay the onset of at least one symptom of a health condition; and to increase the probability that the onset of at least one symptom of a health condition will be delayed.

In some embodiments, administering the therapeutically-effective amount of the neuroplastic agent to the human patient is therapeutically effective to result in neuroplasticity in the human patient. In some specific embodiments, the neuroplasticity treats the condition or a symptom thereof.

In some embodiments, the method comprises identifying that the human patient presents with the condition or a symptom thereof.

In some embodiments, the condition is selected from NAFLD, NASH, cirrhosis of the liver, amyloidosis, a mineral deficiency, a hormonal imbalance, or pineal gland calcification. In some specific embodiments, the condition is selected from NAFLD, NASH, cirrhosis of the liver, and amyloidosis. In some very specific embodiments, the condition is selected from NAFLD, NASH, and cirrhosis of the liver.

In some embodiments, the condition is a mineral deficiency. In some embodiments, the condition is a mineral deficiency, and the mineral is selected from copper, calcium, and magnesium.

In some embodiments, the condition is a hormone imbalance. In some specific embodiments, the condition is melatonin deficiency. In some very specific embodiments, the condition is pineal gland calcification, which results in melatonin deficiency, that presents with insomnia, which results from melatonin deficiency.

In some embodiments, the condition is selected from insomnia, anxiety, depression, post-traumatic stress disorder, and borderline personality disorder.

In some embodiments, the condition is an addiction.

In some embodiments, the prosthetic is selected from a prosthetic leg, a prosthetic arm, a prosthetic foot, a prosthetic hand, a cochlear implant, a bionic eye, a brain-computer interface, a cyborg antenna, a haptic interface, and a magnetic implant.

In some embodiments, the method comprises identifying that the human patient presents with NAFLD, wherein the therapeutically-effective dose of the neuroplastic agent is therapeutically effective to treat the NAFLD in the human patient.

In some embodiments, the method comprises identifying that the human patient presents with NASH, wherein the therapeutically-effective dose of the neuroplastic agent is therapeutically effective to treat the NASH in the human patient.

In some embodiments, the method comprises identifying that the human patient presents with cirrhosis of the liver, wherein the therapeutically-effective dose of the neuroplastic agent is therapeutically effective to treat the cirrhosis of the liver in the human patient.

In some embodiments, the method comprises identifying that the human patient presents with deleterious concentrations of amyloid beta, wherein the therapeutically-effective dose of the neuroplastic agent is therapeutically effective to treat the deleterious concentrations of amyloid beta in the human patient.

In some embodiments, the method comprises identifying that the human patient is deficient in copper, wherein the therapeutically-effective dose of the neuroplastic agent is therapeutically effective to treat symptoms of copper deficiency in the human patient.

In some embodiments, the method comprises identifying that the human patient is deficient in calcium, wherein the therapeutically-effective dose of the neuroplastic agent is therapeutically effective to increase calcium absorption in the human patient.

In some embodiments, the method comprises identifying that the human patient is deficient in magnesium, wherein the therapeutically-effective dose of the neuroplastic agent is therapeutically effective to treat symptoms of magnesium deficiency in the human patient.

In some embodiments, the method comprises identifying that the human patient presents with symptoms caused by pineal gland calcification, wherein the therapeutically-effective dose of the neuroplastic agent is therapeutically effective to treat the symptoms caused by pineal gland calcification.

In some embodiments, the method comprises identifying that the human patient presents with melatonin deficiency, and the therapeutically-effective amount of the neuroplastic agent is therapeutically effective to treat the melatonin deficiency. In some specific embodiments, the method comprises identifying that the human patient presents with melatonin deficiency, the neuroplastic agent is a tryptamine, and the therapeutically-effective amount of the neuroplastic agent is therapeutically effective to treat the melatonin deficiency. In some very specific embodiments, the method comprises identifying that the human patient presents with melatonin deficiency, the neuroplastic agent is psilocybin or psilocin, and the therapeutically-effective amount of the neuroplastic agent is therapeutically effective to treat the melatonin deficiency.

In some embodiments, the method comprises identifying that the human patient presents with deficient fertility, wherein the therapeutically-effective dose of the neuroplastic agent is therapeutically effective to improve the fertility of the human patient.

In some embodiments, the method comprises identifying that the human patient presents with a borderline personality disorder, wherein the therapeutically-effective dose of the neuroplastic agent is therapeutically effective to treat the borderline personality disorder in the human patient.

Various aspects of this disclosure relate to a method to assimilate a human patient to a prosthetic, comprising administering a therapeutically-effective dose of the neuroplastic agent to the human patient, wherein the therapeutically-effective dose of the neuroplastic agent is therapeutically effective to assimilate the human patient to the prosthetic.

In some embodiments, the method comprises identifying that neuroplasticity would enable the human patient to better assimilate to a prosthetic.

In some embodiments, the neuroplastic agent is selected from a tryptamine, an isotryptamine, an imidazopyridine, a benzofuran, a benzothiophene, a fused pyrrolidine, a phenethylamine, an ergoline, a lysergamine, a lysergic acid, an amphetamine, an azepinoindole, a harmala alkaloid, an indole alkaloid, a tropane alkaloid, a 5HT2A-receptor agonist, a 5HT2B-receptor agonist, a 5HT2C-receptor agonist, a 5HTIA-receptor agonist, a serotonin reuptake inhibitor, a NMDA receptor antagonist, and a trace amine-associated receptor 1 agonist.

In some embodiments, the neuroplastic agent is selected from psilocybin, deuterated psilocybin, psilocin, norpsilocin, aeruginascin, baeocystin, norbaeocystin, N,N-dimethyltryptamine (DMT), 5-methoxy-N,N-dimethyltryptamine, 6-fluoro-N,N-diethyltryptamine, 1-((S)-2-aminopropyl)-1H-indazol-6-ol (AL-34662), 3-[(5R)-5-methyl-1,2,5,6-tetrahydropyridin-3-yl]-1H-pyrrolo[2,3-b]pyridine, N-[(2-phenyl)benzyl]-1-(2,5-dimethoxy-4-nitrophenyl)-2-aminoethane (25N-NBPh), lysergic acid diethylamide (LSD), lisuride, JRT, mescaline, 4-iodo-2,5-dimethoxyphenethylamine (2C-I), 4-bromo-2,5-dimethoxyphenethylamine (2C-B), 2,5-dimethoxy-4-iodoamphetamine (DOI), 2,5-dimethoxy-4-bromoamphetamine (DOB), 2,5-dimethoxy-4-chloroamphetamine (DOC), L-DOPA, 25N-N1-Nap, 3,4-methylenedioxymethamphetamine (MDMA), ketamine, harmaline, beta-carboline, lumateperone, ibogaine, noribogaine, tabernanthalog, (2R)-1-(5-methoxy-1H-indol-1-yl)-N,N-dimethylpropan-2-amine (AAZ-A-154), DLX-0001, and DLX-0007. In some specific embodiments, the neuroplastic agent is selected from psilocybin, psilocin, norpsilocin, aeruginascin, baeocystin, norbaeocystin, DMT, LSD, mescaline, MDMA, and ketamine.

In some very specific embodiments, the neuroplastic agent is selected from psilocybin, psilocin, norpsilocin, aeruginascin, baeocystin, norbaeocystin, DMT, LSD, mescaline, and MDMA.

In some embodiments, the neuroplastic agent is a tryptamine. In some specific embodiments, the neuroplastic agent a tryptamine, and the tryptamine is selected from psilocybin, psilocin, norpsilocin, aeruginascin, baeocystin, norbaeocystin, and DMT. In some very specific embodiments, the neuroplastic agent is psilocybin. In some very specific embodiments, the neuroplastic agent is psilocin.

In some embodiments, the method comprises administering multiple doses of the neuroplastic agent to the human patient; the multiple doses of the neuroplastic agent comprise an initial dose, which is administered to the human patient on a first day; the multiple doses of the neuroplastic agent comprise a larger, subsequent dose, which is administered to the human patient on a subsequent day that is subsequent to the first day; the neuroplastic agent displays hallucinogenic side effects; the hallucinogenic side effects of the neuroplastic agent display tachyphylaxis; the initial dose of the neuroplastic agent comprises a first amount of the neuroplastic agent; the larger, subsequent dose of the neuroplastic agent comprises a second amount of the neuroplastic agent that is greater than the first amount of the neuroplastic agent; the first amount of the neuroplastic agent displays a low risk of deleterious hallucinogenic side effects; the second amount of the neuroplastic agent displays a heightened risk of deleterious hallucinogenic side effects; the larger, subsequent dose of the neuroplastic agent is administered after the initial dose of the neuroplastic agent; and administering the larger, subsequent dose of the neuroplastic agent after the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent.

In some embodiments, the larger, subsequent dose of the neuroplastic agent is at least 50 percent greater than the initial dose. In some specific embodiments, the larger, subsequent dose of the neuroplastic agent is at least 100 percent greater than the initial dose. In some very specific embodiments, the larger, subsequent dose of the neuroplastic agent is at least 200 percent greater than the initial dose.

In some embodiments, the neuroplastic agent is selected from psilocybin, psilocin, norpsilocin, aeruginascin, baeocystin, norbaeocystin, and DMT; the first amount is less than 1 milligrams of the neuroplastic agent; and the second amount is greater than 1 milligrams of the neuroplastic agent.

In some embodiments, the neuroplastic agent is selected from psilocybin, psilocin, norpsilocin, aeruginascin, baeocystin, norbaeocystin, and DMT; the first amount is less than 2 milligrams of the neuroplastic agent; and the second amount is greater than 2 milligrams of the neuroplastic agent.

In some embodiments, the neuroplastic agent is selected from psilocybin, psilocin, norpsilocin, aeruginascin, baeocystin, norbaeocystin, and DMT; the first amount is less than 3 milligrams of the neuroplastic agent; and the second amount is greater than 3 milligrams of the neuroplastic agent.

In some embodiments, the neuroplastic agent is LSD; the first amount is less than 5 milligrams of the neuroplastic agent; and the second amount is greater than 5 milligrams of the neuroplastic agent.

In some embodiments, the neuroplastic agent is LSD; the first amount is less than 10 milligrams of the neuroplastic agent; and the second amount is greater than 10 milligrams of the neuroplastic agent.

In some embodiments, the neuroplastic agent is LSD; the first amount is less than 20 milligrams of the neuroplastic agent; and the second amount is greater than 20 milligrams of the neuroplastic agent.

In some embodiments, the larger, subsequent dose of the neuroplastic agent is administered at least 10 hours and no more than 5 days following the administration of the initial dose of the neuroplastic agent. In some specific embodiments, the larger, subsequent dose of the neuroplastic agent is administered at least 12 hours and no more than 72 hours following the administration of the initial dose of the neuroplastic agent. In some very specific embodiments, the larger, subsequent dose of the neuroplastic agent is administered at least 18 hours and no more than 48 hours following the administration of the initial dose of the neuroplastic agent.

In some embodiments, the larger, subsequent dose of the neuroplastic agent is the therapeutically-effective dose of the neuroplastic agent.

In some embodiments, the method comprises administering transcranial magnetic stimulation (TMS) to the human patient after administering the neuroplastic agent. In some specific embodiments, the method comprises administering TMS to the human patient at least 10 minutes and no greater than 10 hours after administering the neuroplastic agent. In some very specific embodiments, the method comprises administering TMS to the human patient at least 60 minutes and no greater than 6 hours after administering the neuroplastic agent.

In some embodiments, the human patient presents with pineal gland calcification; the therapeutically-effective dose of the neuroplastic agent is therapeutically effective to treat the pineal gland calcification; and administering the TMS after administering the neuroplastic agent increases the efficacy of the neuroplastic agent at treating the pineal gland calcification. In some specific embodiments, administering the TMS comprises focusing the TMS on the pineal gland. In some very specific embodiments, administering the TMS comprises focusing the TMS on the pineal gland, and focusing the TMS on the pineal gland after administering the neuroplastic agent increases the efficacy of the neuroplastic agent at treating the pineal gland calcification.

In some embodiments, the human patient presents with amyloidosis of the brain; the therapeutically-effective dose of the neuroplastic agent is therapeutically effective to treat the amyloidosis of the brain; and administering the TMS after administering the neuroplastic agent increases the efficacy of the neuroplastic agent at treating the amyloidosis of the brain. In some specific embodiments, administering the TMS after administering the neuroplastic agent increases the efficacy of the neuroplastic agent at treating the amyloidosis of the brain.

In some embodiments, the method further comprises applying microwaves to heat subterranean elements of a planet, wherein the planet has a core, and the heating is sufficient to mobilize low-molecular weight chemical compounds in the core of the planet such that the low-molecular weight chemical compounds escape the core of the planet and thereby increase the density of the core. In some specific embodiments, the method comprises increasing the density of the core of the planet by at least one femtogram per terameter. In some very specific embodiments, the planet is Mars, and increasing the density of the core of the planet restores the magnetic field on Mars.

In some embodiments, the neuroplastic agent is administered orally.

In some embodiments, the neuroplastic agent is administered by drinking a beverage that comprises the neuroplastic agent. In some specific embodiments, the neuroplastic agent is psilocybin or psilocin, and the neuroplastic agent is administered by drinking a beverage that comprises the neuroplastic agent.

Many variations of the foregoing methods allow for the improved treatment of various conditions, and the Detailed Description that follows contains additional variations. The skilled person will immediately recognize numerous other combinations of the disclosures contained herein, and neither the foregoing Background and Summary nor the Abstract nor the following Detailed Description shall limit any patent claim that matures from this disclosure, which patent claim(s) shall instead be construed in view of the language of the claim(s) as well as their claim dependency and, if any ambiguity remains, then in accordance with the conventional canons of claim construction.

### DESCRIPTION

As amputees adjust to new prosthetics, they experience neuroplasticity-driven cortical remapping, in which neuron connectivity adapts to recognize the replacement of an amputated limb with a prosthetic. The brain literally rewires itself to recognize new sensations and to learn to control the prosthetic.

Psychedelic agents similarly drive neuroplasticity. Cutting-edge research aims to develop and leverage neuroplastic agents to treat various conditions such as anxiety and depression. The development of next-generation neuroplastic agents aims to separate the neuroplastic effects of new pharmaceutical candidates from psychedelic effects to allow therapeutic benefits without psychoactivity.

This disclosure describes the use of neuroplastic agents to accelerate assimilation with a prosthetic. The nature of the human subject and the prosthetic are not limiting. The human subject may be, for example, an amputee, a patient presenting with paralysis, a patient presenting with hearing or vision loss, or a healthy human subject. The prosthetic may be, for example, an artificial limb, a bionic device, a brain-computer interface, or another device that integrates biology with machine.

The principle of this disclosure is simple. When a person engages with a prosthetic that drives neuroplastic change, then the administration of a neuroplastic agent can accelerate that change.

Psilocybin may be used as an effective treatment for indications presented in the above Background and Summary and may be even more effective when used in combination with certain over-the-counter supplements. It is estimated that more than 90 percent of the United States population is deficient in at least one vitamin or mineral according to the recommended Dietary Reference standards. For example, copper, which is normally consumed as part of a well-balanced diet, is involved in brain activities such as memory production and neurotransmitter synthesis. Inadequate levels of copper in the brain contribute to limited neural activation and many mental health indications can be linked to low levels of copper or downstream enzyme concentrations.

Similarly, calcium, is thought to contribute to neuroplasticity and the inability to adequately regulate calcium levels can lead to neural aging and neurodegenerative diseases. The body's stress response releases cortisol and other stress hormones, and this can in turn limit the body's ability to absorb calcium. Psilocybin can reduce stress, anxiety, and other physiological effects of mental health, which can thereby improve mineral regulation, resulting in improved brain function and overall mental health.

Magnesium supplementation is thought to increase brain function in several ways. Magnesium increases serum brain-derived neurotrophic factor (BDNF) in patients with depression, for example, and psilocybin further increases serum BDNF in such patients. Magnesium deficiency can lead to inflammation in the brain as well as neurodegeneration, which can be minimized by consuming psilocybin and other psychedelics.

Many mental health conditions such as post-traumatic stress disorder (PTSD) and depression are linked to low levels of minerals, and it is not clear whether this relationship is causal or correlative. This “vicious cycle” as it is sometimes referred to by researchers, could be interrupted and ultimately stopped by the neuroplasticity action of psychedelics, particularly psilocybin. Psilocybin is therefore an effective treatment for mineral regulation-related mental health indications. Mineral levels should be monitored during the psilocybin treatment process, deficiencies addressed, and supplementation adjusted as needed as mineral levels improve. For this psilocybin plus supplement approach, vitamins and minerals offered as standard solutions or customized cocktails might be administered, which would contain individual-specific amounts of the desired compounds, dependent on the concentrations observed during treatment.

Psychedelics may also improve the function of the pineal gland, reverse calcification, and subsequently increase melatonin levels. It is widely thought that the pineal gland is responsible for the production of melatonin and naturally occurring DMT in the body. Much research has been done to understand the disintegration of this part of the brain, and its mode of action and function remains mostly uncharacterized. Calcium deposits contribute to calcification of the pineal gland, and several environmental factors contribute to this process. For example, ingesting fluorinated compounds in teas and toothpaste, frequent exposure to cell phones or electromagnetic fields, and even drinking tap water is linked to deposits of calcium in the pineal gland region.

Calcification of the pineal gland may be reversed, but the biochemical pathways that reverse calcification remain unclear. When psychedelics are consumed in conjunction with melatonin, however, both the pineal gland and the choroid plexus can be regenerated. Natural production of DMT may be viewed as a form of self-preservation, and consumption of similar psychedelic compounds (i.e., tryptamines) may alleviate damage and restore this portion of the brain.

Beta-nerve growth factor (NGF) levels are linked to fetal development during pregnancy, and higher rates of healthy live births arise from normal levels of NGF. This neurotrophin has been measured in ratios of ProNGF (the precursor form) and NGF at various stages during pregnancy for both mother and fetus. While levels of NGF are typically elevated during each trimester in healthy pregnancies, lower than mean levels of NGF correlate with less successful outcomes including still births.

Anti-sperm antibodies (ASA) are an additional cause of infertility for both women and men, possibly responsible for up to 30 percent of infertile couples. While the cause of ASA remains disputed, current treatments include anti-inflammatory drugs and assisted reproductive techniques such as in vitro fertilization to conceive. ASA production influences both male and female fertility. Since ASA rates are particularly elevated for men who have undergone vasectomy and vasectomy reversals, and ASA can reduce sperm mobility, intracytoplasmic sperm injection may be used to increase pregnancy chances with in vitro fertilization (IVF). Even if the couple conceives, however, ASA-positive individuals are much more likely to experience spontaneous pregnancy loss than those without the antibodies.

Other causes of infertility, including sexual dysfunctions, endometriosis, and high body-mass index can be addressed using neuroplastic agents. Some of these causes are higher for men, while others are more applicable to women. Traditional treatment methods used to overcome these issues often contribute to collateral problems, however, as side effects can cause abnormalities and reduced numbers of live births. One example is the use of sildenafil to treat erectile dysfunction in men, which may improve sperm mobility, but negatively correlates to fertilization and implantation rates.

Melatonin can promote fertility through several ways, including improved ovarian health that leads to regular ovulation and even successful implantation of the fertilized egg into the uterus. Supplementing this naturally occurring hormone has been shown to support both IVF and natural conception rates. Melatonin is produced in the body via a pathway regulated by the pineal gland starting with L-tryptophan and proceeding through a serotonin intermediate. Homeostasis is established between tryptophan and several tryptophan catabolites (TRYCATs) including serotonin, melatonin, tryptamine, and others. Factors that typically influence the various tryptophan pathways are shown below, but prior to this have not considered the impact of tryptamine neuroplastic agents on tryptophan metabolism. The kynurenine pathway is known to give rise to multiple neurological conditions and can even lead to abnormal pregnancies.

Psilocybin and other psychedelics can contribute to hormone regulation by reducing the stress response and subsequent inflammation in patients, while neuroplasticity promotes normal endocrine system functionality and helps restore hormone balance. Hormonal imbalance and endocrine system dysregulation can lead to many physiological disorders, and traditional pharmaceutical treatments can have unpleasant side effects including menopausal symptoms, infertility, acne, abnormal hair growth or loss, hyper- and hypothyroidism, and Addison's and Cushing's disease. Comorbidities with hormone imbalances can additionally limit traditional pharmaceutical treatment options making psychedelics an attractive alternative.

Combining psilocybin with neurotherapy, specifically TMS, may be used to improve both memory production and the retrieval of memories, especially when used in conjunction with mineral supplements such as iron, magnesium, and calcium. TMS is traditionally used to treat neurodegenerative disorders and slow the progression of these diseases by improving neuron firing in targeted portions of the brain. One limitation of TMS is that it can cause seizures, even in patients not previously prone to seizures. The incorporation of psychedelics and other plant medicines such as cannabidiol (CBD) into this treatment is expected to improve efficacy while minimizing negative side effects.

Neuronal excitability may be achieved via TMS, but psychedelic-induced neuroplasticity may also be achieved through similar magnetic stimulation generated from within the body. TMS causes neurons to fire in a series of fast pulsations, which produces electrical current in the brain. This current create a corresponding electromagnetic field. The involvement of an electromagnetic field in the modification of biological processes is not without precedent. Applying a magnetic field to magnetically-modified hepatocyte donor cells, for example, results in superior liver transplant outcomes. Long periods of exposure to magnetic fields have also proven detrimental to the health of the pineal gland, measured by a decrease in the amount of melatonin produced when the field is pulsed in rapid on/off intervals. The mere presence of magnetic fields does not reduce the function of the pineal gland, but introducing and removing a magnetic field in rapid succession results in detrimental effects. TMS should be administered to create action potentials in neurons of one or more targeted regions of the brain or in the brain generally, and the administration of such TMS is well known.

Interestingly, the compound melatonin appears to protect against the negative effects of a low frequency magnetic field. Melatonin contributes to many biological functions across several species of mammals including humans,. Physiological contributions from melatonin include the production of several hormones, improvements in diabetes and diabetes-related neuropathy, reduction of amyloidosis, regulation of calcium channels, and promotion of neurogenesis.

The pineal gland exhibits magnetoreceptivity, which is the ability to sense magnetic fields. Magnetic fields are essential for some biological functions, and the absence of a field results in memory loss and decreased neurogenesis. Magnetoreceptivity is also responsible for migration patterns, and shifts in the earth's naturally occurring magnetic field can result in deviations from usual migration patterns such as when whales and birds appear in areas outside of their seasonal migration paths. The pineal gland is also responsible for synthesis and secretion of melatonin into the cerebrospinal fluid (CSF), which has been shown to balance levels of CSF, which is electrically conductive. Deep, yogic type breathing diplays a 16-28% increase in the power and velocity of CSF flow. Electricity is generated in the body with every heartbeat by electrical impulses in the sinus node pacemaker cells in the upper wall of the right atrium of the heart. The current produced here is circulated throughout the spinal column and brain, generating a bioelectromagnetic field. This field has been measured in long-term meditators, and this electromagnetic field formation from the increased power and velocity of the CSF fluid triggers the production and release of DMT in the brain by the pineal gland.

Meditation and specialized breathing techniques such as the Wimhof Method and holotropic breathing allow individuals to enter altered states of consciousness. However, the mechanism for how these practices lead to visual and hallucinogenic phenomena are not fully understood. Phosphenes, for example, are visual phenomena where light is observed without physically entering the eye. Phosphenes are experienced frequently by meditators and can also be mechanically induced as magnetophosphenes when a person is in the presence of a strong magnetic field. By applying the previously described mechanisms to form strong bioelectromagnetic fields and trigger release of naturally-produced psychoactive compounds in the brain (e.g., DMT), a connection between the healing potentials and neuroplastic effects is observed with psychedelics, particularly when applied in conjunction with electromagnetic fields and supplements such as melatonin.

Decalcification of the pineal gland and choroid plexus leading to increased levels of melatonin or supplementation with melatonin and daily doses of psychedelics may contribute to the generation of a bioelectromagnetic field while simultaneously shielding from external and potentially detrimental electric and magnetic fields. Not only has the presence of low frequency fields increased in our environment, but researchers have shown humans are physiologically and psychologically susceptible to the low frequency shifts of the earth's magnetic field. Frequency shifts of the earth's magnetic field may even trigger episodes of psychosis and seizures, and such occurrences are expected to rise during periods of weakening magnetic field strength as part of a pending magnetic pole shift.

Neuroplastic agents when taken in conjunction with GLP-1 receptor agonists, could help alleviate symptoms of NAFLD, NASH, and related conditions. Neuroplastic agents may also directly impact insulin resistance with minimal impact on the liver and other organs.

Amyloid beta is a common cause of encephalopathy in patients with NAFLD. The reduction of peripheral amyloid beta can minimize cognitive impairment in patients with NAFLD. Similarly, Alzheimer's patients display high levels of amyloid beta. High concentrations of amyloid beta in the brain correlates with the development of Alzheimer's Disease and the deterioration of cognitive function including memory and fine motor skills. Neuroplastic agents generally and 5-hydroxytryptamine receptor agonists specifically enhance gamma-frequency oscillations and help clear amyloid build-up thereby improving cognitive function. Inflammation in the brain may also be treated with neuroplastic agents.

Neuroplastic agents alleviate the progression of NAFLD in part through the regulation of the production of cytokines through 5-HT1A, 5-HT2A, 5-HT2B, and 5-HT2C receptor pathways thereby controlling inflammatory response. Neuroplastic agents also activate other G-protein coupled receptors (GPCR) to reduce inflammation and modulate cell proliferation, and they also modulate dipeptidyl peptidase-4 (DPP-4) functionality and thiazolidinedione action on glucose and fatty acids, which is beneficial for treating liver disease generally and NAFLD specifically. Neuroplastic agents also activate SIRT1, which can treat NAFLD.

In a similar way in which psychedelics impact memories and result in neuroplasticity, psilocybin and other psychedelics can treat the different recognized forms of borderline personality disorders (BPDs) and address the downstream effects of mental processing disorders such as anger and addiction. Although symptoms such as anger, aggression, and addiction may be considered manifestations of underlying mental health disorders, symptoms such as these are often observed across a wide range of indications and may not be fully understood or appropriately diagnosed or treated. The use of psychedelics may also treat physiological causes such as trauma to the brain and promote healing or help lay new patterns of learned behaviors and responses to various triggers.

Many neuroplastic agents are known. The nature of the neuroplastic agent is not limiting. Naturally-occurring neuroplastic agents include psychedelics including psilocin (the active form of the prodrug psilocybin), DMT, mescaline, and LSD. Psilocin and DMT are both tryptamines that bind 5-hydroxytryptamine (5HT) receptors in the brain such as 5HT2A receptor. Mescaline is a phenethylamine that binds to 5HT receptors as well as adrenergic receptors. Dopamine, L-DOPA, and epinephrine are other examples of phenethylamines. LSD is a lysergamide, which binds both 5HT receptors and dopamine receptors.

Numerous analogs of tryptamines, phenethylamines, and lysergamides have been synthesized as cataloged, for example, by Dr. Alexander Shuglin, who published synthetic routes, dosing, and his personal psychedelic experiences in PIHKAL: A CHEMICAL LOVE STORY and TIHKAL: THE CONTINUATION, which are incorporated by reference in their entireties. As psychedelic effects correlate with neuroplasticity, Dr. Shuglin's publications provide invaluable insights into the genus of neuroplastic agents as well as their dosing. Other neuroplastic agents are described, for example, in U.S. Patent Application No. 2023/0295106 A1, U.S. Patent Application No. 2023/0219969 A1, U.S. Patent Application No. 2023/0227453 A1, International Application Publication No. WO 2023/114325 A1, International Application Publication No. WO 2023/114313 A1, International Application Publication No. WO 2023/114320 A1, International Application Publication No. WO 2023/114858 A1, International Application Publication No. WO 2023/114844 A1, International Application Publication No. WO 2023/114238 A1, and International Application Publication No. WO 2022/241006 A1, each of which is incorporated by reference in its entirety.

Anecdotal evidence suggests that animals can detect human diseases and medical alert service animals are becoming useful companions for individuals with disorders such as diabetes, epilepsy, and anxiety disorders. Canines' extraordinary sense of smell enables them to detect unique volatile organic compounds (VOCs) that are thought to be released moments prior to seizures. Canine magnetoreceptivity may also interpret changes in human bioelectromagnetic fields that correlate with a wide range of neurological conditions. Understanding this mechanism of action and the role that these animals play in altering our brain chemistry can lead to superior training and detection and also alleviate many of the symptoms experienced by these disabled individuals.

While psychoactive dosages of neuroplastic agents generally result in neuroplasticity, sub-psychoactive dosages (informally referred to as micro-doses) can beneficially result in neuroplastic effects while minimizing or eliminating psychedelic side effects. Additionally, the development of next-generation neuroplastic agents aims to divorce psychedelic and neuroplastic effects, for example, by altering known tryptamine and phenethylamine scaffolds to target receptors that drive neuroplasticity but not psychoactivity. Dr. David E. Olson co-founded Delix Therapeutics, for example, to commercialize the indole alkaloid tabernanthalog and the isotryptamine AAZ-A-154 and to develop a catalog of other analogs of neuroplastic agents to treat various neurological conditions.

A different strategy to minimize psychedelic effects is simply to gradually increase dosage over time. Repeated dosing of psychedelic neuroplastic agents generally creates tolerance to their hallucinogenic effects while nevertheless retaining neuroplastic effects. A dosing regimen for LSD and pure psilocybin follows in Table 1.

TABLE 1
Example Loading Doses for LSD and Psilocybin
to Minimize Hallucenogenic Side Effects

| | LSD dose | Psilocybin dose |
|---|---|---|
| Day | (micrograms) | (milligrams) |
| 1 | 2.5 | 0.625 |
| 2 | 5 | 1.25 |
| 3 | 10 | 2.5 |
| 4 | 20 | 5 |
| 5+ | 40 | 10 |

As depicted in Table 1, the dosing of a neuroplastic agent varies greatly depending upon the compound. In some embodiments, a unit dose of the neuroplastic agent is at least 5 nanograms and no greater than 500 milligrams. In some specific embodiments, a unit dose of the neuroplastic agent is at least 5 nanograms and no greater than 500 nanograms. In some specific embodiments, a unit dose of the neuroplastic agent is at least 50 nanograms and no greater than 5 micrograms (such as a micro-dose of LSD). In some specific embodiments, a unit dose of the neuroplastic agent is at least 500 nanograms and no greater than 50 micrograms (such as the LSD dosing set forth in Table 1). In some specific embodiments, a unit dose of the neuroplastic agent is at least 5 micrograms and no greater than 500 micrograms. In some specific embodiments, a unit dose of the neuroplastic agent is at least 50 micrograms and no greater than 5 milligrams (such as a micro-dose of psilocybin). In some specific embodiments, a unit dose of the neuroplastic agent is at least 500 micrograms and no greater than 50 milligrams (such as the psilocybin dosing set forth in Table 1). In some specific embodiments, a unit dose of the neuroplastic agent is at least 5 milligrams and no greater than 500 milligrams (such as a standard dosage of ketamine, MDMA, or lumateperone). Additional dosing information may be found, for example, in PiHKAL, TiHKAL, at Erowid.com, and at ClinicalTrials.gov, each of which is incorporated by reference in its entirety.

The frequency of a unit dose is not limiting as neuroplastic agents display neuroplastic properties following a single dose. Multiple doses might nevertheless be desirable, for example, to build tolerance to hallucinogenic side effects such as the dosing regimens depicted in Table 1. Multiple doses might also be desirable to drive more significant neuroplastic change. Dose frequency may vary, for example, from twice daily to annually such as daily, weekly, monthly, or annually.

The dose may be administered prior to use of the prosthetic, but the timing of the dose is not limiting. Some neuroplastic agents such as phenethylamines and lysergamides that bind adrenergic receptors are central nervous system stimulants with a duration on the order of several hours or more. Such neuroplastic agents might be administered in the morning, for example, to minimize the risk of insomnia.

The prosthetic itself is not limiting. In some embodiments, the prosthetic is selected from a prosthetic leg, a prosthetic arm, a prosthetic foot, a prosthetic hand, a cochlear implant, a bionic eye, a brain-computer interface, a cyborg antenna, a haptic interface, or a magnetic implant.

Suitable carriers for dosing psilocybin are depicted in the table below, altering both the time of onset and the absorption potential of the medicine. Lighter molecular weight psychoactive compounds may alternatively be dosed, for example, by inhalation.

TABLE 2

| Carrier | pH |
|---|---|
| Water | 7-7.5 |
| Kombucha tea | 2.4 |
| Sparkling wines | 2.9 |
| Red wines | 3.5-3.8 |
| White wines | 3.1-3.4 |
| Bourbon | 4.67 |
| Cream | 6.1-6.7 |
| Mint teas | 6.4-7.2 |

Nikola Tesla recognized the significance of the pyramids in Egypt and suspected they were giant transmitters of energy. Knowing they were made of conductive materials such as limestone and granite, he proposed they may play a role in transmitting wireless energy, also noting their dimensions, location, and alignment with the Earth's magnetic field. He attempted to build Wardenclyffe Tower on Long Island to replicate these energetic principles. Although, he was unsuccessful in demonstrating his theories of wireless energy on a large scale, many advances and discoveries have been made in the decades since his work.

One such discovery is the naturally occurring resonant frequencies of Earth called the Schumann Resonance frequencies. Winfried Schumann was brought to the United States from Germany at the end of WWII via the secret “Operation Paperclip” and, in 1952, he proposed a frequency range of 3-60 Hz for Earth's resonant frequencies. Many researchers have since refined the mathematical equations to approximate their modes and measurement stations for these extremely low frequency (ELF) waves have been incorporated at several locations around the globe. These waves are produced when lightning excites the resonant cavity between the conductive ionosphere and the Earth's surface.

Recent claims that cylinder-shaped wells or shafts were discovered under at least one of the pyramids in Egypt (i.e., the Khafre Pyramid) provides some support for Tesla's theories that the pyramids may support energy generation. It has also been shown that the largest pyramid in the Giza complex can concentrate electromagnetic energy, particularly in its internal and underground chambers, mostly successfully for wavelengths between 200 and 250 m. There are also claims of a similar pyramid structure in the mountains of Visoko, Bosnia that emits an energetic beam of 28 kHz in the radiowave spectrum. Underground structures have been shown to exist for this pyramid as well, and the concentration of negative ionization levels in these chambers is significant. Some suspect that the pyramids in Egypt are not functioning in the way they were meant to, especially compared to the energetic observations of similar pyramids in other parts of the world. One theory that may explain its failure is that the land in the Giza complex was once covered in water. This theory is supported by the erosion patterns seen on the Great Sphinx. Also, Pliny the Elder, who wrote about the pyramids in the first century in Rome, suggested the pyramids were erected using large amounts of salt which were washed away with water. He also noted that the pyramid in question had a deep well underneath. Interestingly, when the wave frequency is calculated from a 200 m wavelength (as suggested in the study referenced above), for an assumed wave velocity of 1,563 m/s for soundwaves in seawater at 40° C., the wave frequency is 7.8Hz, the same frequency as mode 1 of the Schumann Resonance frequency.

The dimensions of one side of the base of the Giza pyramid is approximately 230 m and its dimensions are relevant to the wavelength of the resonant frequency of the Earth. If the area around the pyramids was covered in warm saltwater, it is suggested that an alignment with the naturally occurring resonant frequencies of the Earth would be magnified according to the Energy Wave Theory (EWT) and even allow for energy generation and interaction with the Earth's ionosphere. This is presented as the missing link to the wireless energy potential of the Egyptian pyramids that Tesla and many others have proposed.

To provide further support for this theory, many of the smaller pyramids in the Giza complex have dimensions that correlate to the other modes of Schumann resonant frequencies of the Earth, as shown in the table below. It is interesting to note that the size of the many pyramids in the Giza complex appear to be clustered in these correlated dimensions, suggesting the ancient Egyptians understood that multiple frequency modes existed and added onto the electricity generating potential across generations. Although there may not be an exact correlation observed for the Schumann frequency modes, it is important to note that mode 1 foundational frequencies vary across a range of 7-16 Hz, so having slight fluctuations in the correlating pyramidic oscillator/amplifiers could prove valuable.

TABLE 3

| | | | Soundwave | |
|---|---|---|---|---|
| | Side | | frequency in | Schumann |
| | dimension | Wavelength | seawater @ | Frequency |
| Pyramid | (m) | (m) | 40C | modes 1-5 |
| Khafra | 215 | 200 | 7.815 | 7.83 |
| Djoser | 109 | 110 | 14.2 | 14.2 |
| Sahure | 78.75 | 78 | 20.03 | 20.03 |
| Unas, Neferefre | 57.75, 65 | 61 | 25.6 | 25.9 |
| Amenemhat II | 50 | 49 | 31.9 | 32 |

The Schumann Resonance frequency of 7.83 Hz is not only relevant to the earth's electromagnetic field and the potential for generating abundant, wireless energy, it also has profound impacts on the human brain. The brain operates in five main frequencies, as shown in the Table 4 below.

TABLE 4

| | | | Schumann | Schumann |
|---|---|---|---|---|
| Brainwave | Brainwave | | resonance | resonance |
| Type | frequency | | mode (s) | frequency |
| Delta | 1-3 | Hz | Mode 1 | 7.83 |
| Theta | 4-7 | Hz | Mode 1 | 7.83 |
| Alpha | 8-12 | Hz | Mode 1, 2 | 7.83, 14.2 |
| Beta | 13-26 | Hz | Modes 2, 3, and 4 | 14.2, 20.03, 25.9 |
| Gamma | 27+ | Hz | Mode 5 and higher | 32 |

Many have noted the overlap of the Schumann frequencies and common brainwave frequencies; however, this disclosure suggests that as the power and intensity of these fundamental frequencies are amplified, the general improvement of many mental and physical health conditions will follow. Mental health conditions have been on the rise, particularly for younger generations, and at the same time, our environments increasingly surrounding us with damaging electromagnetic fields and frequencies that can overpower the supportive frequencies of the earth.

The race to send the first humans to Mars is currently being funded by some of the wealthiest people on Earth. Elon Musk has set Space X goals to send the spacecraft Starship to Mars as early as 2026 and to send people to the red planet by 2029. Mars displays some of the most interesting topography in the solar system. Its tallest peak is a volcano that is 17 miles high and 370 miles in diameter. It also has lightning storms that generate Schumann Resonances frequencies similar to the those experienced on Earth, although they are weaker in intensity and have a broader range.

It is thought that electromagnetic fields like the one around Earth also existed at one time on the planets Mars, Venus, and the Earth's moon. Earth's electromagnetic field enables life by attenuating the impact of solar winds. These solar winds are given off at varying degrees during the sun's twelve-year cycle and most frequently during the period of its magnetic pole reversal, diminish planetary atmospheres, particularly when the planet lacks a strong magnetic field. Without stable atmospheres, Mars, Venus, and Earth's moon are currently considered uninhabitable. Due to their proximity to Earth and to the Sun, Mars, Venus, and the Earth's moon would be the primary candidates of interest for inhabitation by humans. The restoration and regeneration of planetary electromagnetic fields is proposed here, and once regenerated, life may be sustained on these planets.

Earth's magnetic field is likely generated by the electrical current produced by the continual flow of molten nickel and iron within its core. This “dynamo” theory can also be roughly applied to Jupiter, Saturn, Uranus, and Neptune, which experience electromagnetic fields from molecular hydrogen or ionic salts instead of iron. Fast rotation of these spheres causes intense heat at the core, which contributes to the fluidity and movement of the ionic particles, generating electric current and resulting in an electromagnetic field. Although Mars has areas of weak electromagnetic field, it is very low in strength, and leaves it unprotected from solar winds and solar storms. It is thought that Mars may have lost its magnetic field in a large collision of plasma released from the sun, in an event that left behind the “scar” Valles Marineris. This gash in the surface of Mars is six miles deep and almost 3,000 miles in length. Geographically, it is close to the western volcanic region and some researchers have theorized that an impact of this magnitude may have allowed for cracking and shifting of layers deep within the planet's core.

If the regeneration of a strong electromagnetic field will help to restore the atmosphere on the planet Mars, and the necessary magnetic materials are already thought to exist within the planets core, then the gap appears to be the molten core. The circumference of Mars is smaller than Earth's, and it is not quite spherical, but rather an oblate spheroid. It is therefore about 13,200 miles pole-to-pole and 13,300 miles around the equator. Its mass is about 10 times less than Earth's and its gravitational pull is only about 38 percent relative to Earth's. The gravitational pull is likely to change with the reintroduction of a magnetic field, however, as described by Einstein's theory of gravity. It is thought that Mars has an inner core similar to Earth's, although possibly somewhat lighter in density, and may contain a few lighter elements such as sulfur and oxygen. This core, however, is shielded by an insulting layer of rock and silica, which are thought to prevent the heating and cooling cycles needed to create the appropriate fluid movement to generate electric current. These could be the lighter and less dense materials that may have been able to penetrate the surface of the planet during the proposed plasma impact event referenced previously.

It is proposed here that by applying microwaves, (in a frequency set to preferentially target the elements below Mars surface) in a design that mimics a household microwave and magneto tube, it will preferentially heat these subterranean elements. The lowest points of the planet such as the Valles Marineris (six miles below the surface), would represent good locations for these microwave setups. By selectively applying heat in the form of microwave energy, subterranean pressure may be increased and released via the many volcanos in this geographical region. Olympus Mons, which is the tallest known volcano in the solar system, stands over 13 miles tall and is only 1483 miles away from the northwestern edge of Valles Marineris. Researchers have shown that the magma beds that fuel Mars' volcanos run much deeper than those on Earth, which will allow the release of lighter weight components deep below Mars surface, thereby restoring its dense core structure and electromagnetic field.

The Schumann Resonance frequencies are different on Mars than on Earth, and the foundational resonance frequencies on the planet Mars can be estimated using Equation 1 below. These frequencies have been proposed recently by researchers to be in the range of 9-14 Hz. Several factors contribute to this calculation, including c as the velocity of light in a vacuum, the size of Mars given as the radius (R), the thickness of the cavity d between the planet's surface and the lower portion of the ionosphere, and other relative permittivity and conductivity components.

$${f}_{n} \approx \frac{c}{2 π R} \sqrt{n ( n + 1 ) \frac{1 - \frac{d}{R}}{{ε}_{r} ( 1 + i \frac{σ}{{ε}_{r} {ε}_{o} 2 π {f}_{n}} )}} \qquad Eq . 1$$

The atmosphere of Mars is composed primarily of carbon dioxide (>95%), atmospheric pressure is less than one percent of Earth's, and frequent dust storms fill the atmosphere with charged particles. Restoration of the magnetic field on Mars will change the atmosphere. Many studies link changes in the Earth's atmosphere to shifts in our magnetic field over the past decades, which allows the approximation of the effect of restoring the magnetic field on Mars on its atmosphere. This would correspondingly shift the extremely low frequencies (ELF) observed on the planet to date.

When calculating the Schumann Resonance frequencies on Mars, the smaller diameter of the planet reduces capacitance to about 0.000377 Farad (F) from the capacitance of 0.000711 F observed on Earth. Presuming that Mars displays approximately the same inductance of 1 Henries (H) for Earth, then the expected foundational resonance frequency on Mars would be approximately 10 Hz. Additionally, a large pyramid shaped structure has been photographed on the surface of Mars, which imitates the mathematical geometries of the Giza pyramids on Earth. This pyramid, however, is massive in comparison. Image analysis suggests dimensions of 5.06 km per side and a 20,238 m perimeter. Its estimated height is 3.22 km and represents the same dimensional relationship to x as observed in the Giza pyramids. Applying the same principles presented for amplifying the foundational resonances on Earth to this pyramid on Mars tends to suggest that introducing wavelengths of approximately 500 m would result in a wave frequency of about 10 Hz, assuming this pyramidal structure consists of some type of solid rock similar to the surface of Mars, and may have wave velocities around 5000 m/s. Amplification of the resonant frequencies on Mars could thereby allow this pyramid to generate electricity.

The phrases “some embodiments,” “some specific embodiments, “some even more specific embodiments,” and “some very specific embodiments” differentiate more generic embodiments from more specific embodiments, and no other meaning shall be ascribed to the four foregoing phrases.

What is claimed is:

1. A method to treat a condition in a human patient, comprising:
identifying that the human patient presents with a condition selected from non-alcoholic fatty liver disease (NAFLD), nonalcoholic steatohepatitis (NASH), cirrhosis of the liver, and amyloidosis; and
administering a therapeutically-effective dose of a neuroplastic agent to the human patient, wherein the therapeutically-effective dose of the neuroplastic agent is therapeutically effective to treat the condition or a symptom thereof, wherein:
the neuroplastic agent is psilocybin;
the method comprises administering multiple doses of the neuroplastic agent to the human patient;
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount is less than 2 milligrams of the psilocybin;
the second amount is greater than 2 milligrams of the psilocybin;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent;
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent; and
the larger, subsequent dose of the neuroplastic agent is the therapeutically-effective dose of the neuroplastic agent.

2. A method to treat a condition in a human patient, comprising:
identifying that the human patient presents with a condition selected from non-alcoholic fatty liver disease (NAFLD), nonalcoholic steatohepatitis (NASH), cirrhosis of the liver, amyloidosis, a mineral deficiency, a hormonal imbalance, and pineal gland calcification; and
administering a therapeutically-effective dose of a neuroplastic agent to the human patient, wherein the therapeutically-effective dose of the neuroplastic agent is therapeutically effective to treat the condition or a symptom thereof, wherein:
the neuroplastic agent is selected from psilocybin, psilocin, norpsilocin, aeruginascin, baeocystin, norbaeocystin, N,N-dimethyltryptamine (DMT), lysergic acid diethylamide (LSD), mescaline, and 3,4-methylenedioxymethamphetamine (MDMA);
the method comprises administering multiple doses of the neuroplastic agent to the human patient;
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent;
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent; and
the larger, subsequent dose of the neuroplastic agent is the therapeutically-effective dose of the neuroplastic agent.

3. A method to treat a condition in a human patient, comprising:
identifying that the human patient presents with the condition; and
administering a therapeutically-effective dose of a neuroplastic agent to the human patient, wherein the therapeutically-effective dose of the neuroplastic agent is therapeutically effective to treat the condition or a symptom thereof.

4. The method of claim 3, comprising one of:
identifying that neuroplasticity would enable the human patient to better assimilate to a prosthetic, wherein the therapeutically-effective dose of the neuroplastic agent is therapeutically effective to assimilate the human patient to the prosthetic;
identifying that the human patient presents with non-alcoholic fatty liver disease (NAFLD), wherein the therapeutically-effective dose of the neuroplastic agent is therapeutically effective to treat the NAFLD in the human patient;
identifying that the human patient presents with nonalcoholic steatohepatitis (NASH), wherein the therapeutically-effective dose of the neuroplastic agent is therapeutically effective to treat the NASH in the human patient;
identifying that the human patient presents with cirrhosis of the liver, wherein the therapeutically-effective dose of the neuroplastic agent is therapeutically effective to treat the cirrhosis of the liver in the human patient;
identifying that the human patient presents with deleterious concentrations of amyloid beta, wherein the therapeutically-effective dose of the neuroplastic agent is therapeutically effective to treat the deleterious concentrations of amyloid beta in the human patient;
identifying that the human patient is deficient in copper, wherein the therapeutically-effective dose of the neuroplastic agent is therapeutically effective to treat symptoms of copper deficiency in the human patient;
identifying that the human patient is deficient in calcium, wherein the therapeutically-effective dose of the neuroplastic agent is therapeutically effective to increase calcium absorption in the human patient;
identifying that the human patient is deficient in magnesium, wherein the therapeutically-effective dose of the neuroplastic agent is therapeutically effective to treat symptoms of magnesium deficiency in the human patient;
identifying that the human patient presents with symptoms caused by pineal gland calcification, wherein the therapeutically-effective dose of the neuroplastic agent is therapeutically effective to treat the symptoms caused by pineal gland calcification;
identifying that the human patient presents with deficient fertility, wherein the therapeutically-effective dose of the neuroplastic agent is therapeutically effective to improve the deficient fertility of the human patient; and
identifying that the human patient presents with a borderline personality disorder, wherein the therapeutically-effective dose of the neuroplastic agent is therapeutically effective to treat the borderline personality disorder in the human patient.

5. The method of claim 3, wherein:
the therapeutically-effective dose of the neuroplastic agent is therapeutically effective to assimilate the human patient to a prosthetic; and
the prosthetic is selected from a prosthetic leg, a prosthetic arm, a prosthetic foot, a prosthetic hand, a cochlear implant, a bionic eye, a brain-computer interface, a cyborg antenna, a haptic interface, and a magnetic implant.

6. The method of claim 3, wherein the neuroplastic agent is selected from a tryptamine, an isotryptamine, an imidazopyridine, a benzofuran, a benzothiophene, a fused pyrrolidine, a phenethylamine, an ergoline, a lysergamine, a lysergic acid, an amphetamine, an azepinoindole, a harmala alkaloid, an indole alkaloid, a tropane alkaloid, a 5HT2A-receptor agonist, a 5HT2B-receptor agonist, a 5HT2C-receptor agonist, a 5HTIA-receptor agonist, a serotonin reuptake inhibitor, a NMDA receptor antagonist, and a trace amine-associated receptor 1 agonist.

7. The method of claim 3, wherein the neuroplastic agent is selected from psilocybin, deuterated psilocybin, psilocin, norpsilocin, aeruginascin, baeocystin, norbaeocystin, N,N-dimethyltryptamine (DMT), 5-methoxy-N,N-dimethyltryptamine, 6-fluoro-N,N-diethyltryptamine, 1-((S)-2-aminopropyl)-1H-indazol-6-ol (AL-34662), 3-[(5R)-5-methyl-1,2,5,6-tetrahydropyridin-3-yl]-1H-pyrrolo[2,3-b]pyridine, N-[(2-phenyl)benzyl]-1-(2,5-dimethoxy-4-nitrophenyl)-2-aminoethane (25N-NBPh), lysergic acid diethylamide (LSD), lisuride, JRT, mescaline, 4-iodo-2,5-dimethoxyphenethylamine (2C-I), 4-bromo-2,5-dimethoxyphenethylamine (2C-B), 2,5-dimethoxy-4-iodoamphetamine (DOI), 2,5-dimethoxy-4-bromoamphetamine (DOB), 2,5-dimethoxy-4-chloroamphetamine (DOC), L-DOPA, 25N-N1-Nap, 3,4-methylenedioxymethamphetamine (MDMA), ketamine, harmaline, beta-carboline, lumateperone, ibogaine, tabernanthalog, (2R)-1-(5-methoxy-1H-indol-1-yl)-N,N-dimethylpropan-2-amine (AAZ-A-154), DLX-0001, and DLX-0007.

8. The method of claim 3, wherein the neuroplastic agent is selected from psilocybin, psilocin, norpsilocin, aeruginascin, baeocystin, norbaeocystin, N,N-dimethyltryptamine (DMT), lysergic acid diethylamide (LSD), mescaline, 3,4-methylenedioxymethamphetamine (MDMA), and ketamine.

9. The method of claim 3, comprising administering multiple doses of the neuroplastic agent to the human patient, wherein:
the neuroplastic agent is psilocybin;
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount is less than 2 milligrams of the psilocybin;
the second amount is greater than 2 milligrams of the psilocybin;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent; and
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent.

10. The method of claim 3, comprising administering multiple doses of the neuroplastic agent to the human patient, wherein:
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent; and
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent.

11. The method of claim 3, comprising administering transcranial magnetic stimulation (TMS) to the human patient at least 10 minutes and no greater than 10 hours after administering the neuroplastic agent, wherein:
the human patient presents with pineal gland calcification;
the therapeutically-effective dose of the neuroplastic agent is therapeutically effective to treat the pineal gland calcification;
administering the TMS comprises focusing the TMS on the pineal gland; and
focusing the TMS on the pineal gland at least 10 minutes and no greater than 10 hours after administering the neuroplastic agent increases the efficacy of the neuroplastic agent at treating the pineal gland calcification.

12. The method of claim 3, comprising administering transcranial magnetic stimulation (TMS) to the human patient at least 10 minutes and no greater than 10 hours after administering the neuroplastic agent, wherein:
the human patient presents with amyloidosis of the brain;
the therapeutically-effective dose of the neuroplastic agent is therapeutically effective to treat the amyloidosis of the brain; and
the TMS increases the efficacy of the neuroplastic agent at treating the amyloidosis of the brain.

13. The method of claim 3, comprising administering transcranial magnetic stimulation (TMS) to the human patient at least 10 minutes and no greater than 10 hours after administering the neuroplastic agent.

14. The method of claim 3, further comprising applying microwaves to heat subterranean elements of a planet, wherein the planet has a core, and the heating is sufficient to mobilize low-molecular weight chemical compounds in the core of the planet such that the low-molecular weight chemical compounds escape the core of the planet and thereby increase the density of the core.

15. The method of claim 14, comprising increasing the density of the core of the planet by at least one femtogram per terameter.

16. The method of claim 3, wherein:
the neuroplastic agent is psilocybin or psilocin; and
the neuroplastic agent is administered by drinking a beverage that comprises the neuroplastic agent.

17. The method of claim 3, comprising identifying that the human patient presents with melatonin deficiency, wherein:
the neuroplastic agent is psilocybin or psilocin; and
the therapeutically-effective amount of the neuroplastic agent is therapeutically effective to treat the melatonin deficiency.

18. The method of claim 3, comprising administering multiple doses of the neuroplastic agent to the human patient, wherein:
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount is less than 2 milligrams of the neuroplastic agent;
the second amount is greater than 2 milligrams of the neuroplastic agent;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent;
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent;
the condition is selected from non-alcoholic fatty liver disease (NAFLD), nonalcoholic steatohepatitis (NASH), and cirrhosis of the liver; and
the neuroplastic agent is psilocybin or psilocin.

19. The method of claim 3, comprising administering multiple doses of the neuroplastic agent to the human patient, wherein:
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount is less than 2 milligrams of the neuroplastic agent;
the second amount is greater than 2 milligrams of the neuroplastic agent;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent;
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent;
the condition is selected from insomnia, anxiety, depression, post-traumatic stress disorder, and borderline personality disorder; and
the neuroplastic agent is psilocybin or psilocin.

20. The method of claim 3, comprising administering multiple doses of the neuroplastic agent to the human patient, wherein:
the multiple doses of the neuroplastic agent comprise an initial dose, which is administered to the human patient on a first day;
the multiple doses of the neuroplastic agent comprise a larger, subsequent dose, which is administered to the human patient on a subsequent day that is subsequent to the first day;
the neuroplastic agent displays hallucinogenic side effects;
the hallucinogenic side effects of the neuroplastic agent display tachyphylaxis;
the initial dose of the neuroplastic agent comprises a first amount of the neuroplastic agent;
the larger, subsequent dose of the neuroplastic agent comprises a second amount of the neuroplastic agent that is 50 percent greater than the first amount of the neuroplastic agent;
the first amount is less than 2 milligrams of the neuroplastic agent;
the second amount is greater than 2 milligrams of the neuroplastic agent;
the first amount of the neuroplastic agent displays a low risk of deleterious hallucinogenic side effects;
the second amount of the neuroplastic agent displays a heightened risk of deleterious hallucinogenic side effects;
the larger, subsequent dose of the neuroplastic agent is administered at least 10 hours and no more than 5 days following the administration of the initial dose of the neuroplastic agent;
administering the larger, subsequent dose of the neuroplastic agent at least 10 hours and no more than 5 days following the administration of the initial dose of the neuroplastic agent results in tachyphylaxis of the hallucinogenic side effects of the neuroplastic agent such that the initial dose attenuates the heightened risk of deleterious hallucinogenic side effects of the second amount of the neuroplastic agent;
the condition is an addiction; and
the neuroplastic agent is psilocybin or psilocin.
